(19) United States
(12) Patent Application Publication
(10) Pub. No.: US 2006/0195171 A1
(43) Pub. Date: Aug. 31, 2006

(54) Stabilizing apparatus for use with implantable leads

(75) Inventors: John Greenland, San Diego, CA (US); Drew Blankenship, Lake Elsinore, CA (US); David Anderson, Temecula, CA (US); Paul Haldeman, Murrieta, CA (US); Jim Niederecker, Temecula, CA (US); Nathalie Quiles, Murrieta, CA (US)

Correspondence Address: HOLLINGSWORTH & FUNK, LLC, Suite 125, 8009 34th Avenue South, Minneapolis, MN, 55425, US

(73) Assignee: Cardiac Pacemakers, Inc., St. Paul, MN (US)

(21) Appl. No.: 11/399,078

(22) Filed: Apr. 6, 2006

Related U.S. Application Data
(62) Division of application No. 10/465,517, filed on Jun. 19, 2003.

(51) Int. Cl. A61N 1/05

(52) U.S. Cl. 607/119; 607/9

(57) ABSTRACT

A method of placing an implantable device in a cardiac vessel involves positioning the implantable device so that a distal end of the implantable device is positioned in the cardiac vessel. A stylet having an enlarged distal portion is positioned within a lumen of the implantable device so that the enlarged distal portion of the stylet is located at a distal portion of the implantable device. A sleeve is positioned within the implantable device and in proximity to the enlarged distal portion of the stylet. The implantable device is stabilized by selective engagement and disengagement between the enlarged distal portion of the stylet and the sleeve.

### RELATED PATENT DOCUMENTS

This application is a divisional of U.S. patent application Ser. No. 10/465,517 filed on Jun. 19, 2003, which is incorporated herein by reference in its entirety.

### FIELD OF THE INVENTION

The invention relates generally to implantable medical devices, and more particularly devices used for securing of medical devices during implantation procedures.

### BACKGROUND OF THE INVENTION

Devices such as implantable cardioverter/defibrillators (ICD) and pacemakers are well known and effective devices for treating patients with conditions such as cardiac rhythmic dysfunction. The ICD or pacing system usually consists of a pulse generator and one or more electrically conductive leads used for sensing or shocking. The implantation procedure generally takes about two hours and is relatively low risk, as it rarely requires open heart surgery. Usually, one to two leads are placed through a large vein in the chest and threaded down to the inside of the heart. The leads are then connected to the pulse generator, which is placed in a pocket under the skin of the patient.

One common element of both ICD and pacemaker systems that are used to treat congestive heart failure is a pacing lead that is placed on the free wall of the left ventricle within the venous anatomy. The details of the implantation procedure for this lead vary depending on the condition and anatomy of the patient, but typically a guiding catheter is introduced through a major blood vessel such as the subclavian vein. The catheter is then moved through the vasculature (e.g. into the superior vena cava) to locate and access the vessel of interest in the heart, such as the coronary sinus. The catheter can either be navigated on its own through the vasculature to the ostium of the coronary sinus or can be slid over a previously inserted guide wire. After the coronary sinus ostium has been located by the guiding catheter, a lead can be inserted through the catheter into the coronary sinus or one of its branches.

After the lead is successfully implanted, the guide catheter must be removed from the patient. This removal operation creates a risk of dislodging the lead because of the forces applied by the retracting catheter against the lead. Needless to say, dislodging the lead would be problematic, as it requires additional time to reseat the lead. Any such time added to the procedure would be, of course, undesirable, and always carries a risk of the therapy not being delivered during the second attempt.

There is a need for a way of temporarily securing a newly implanted lead while apparatus used in the implantation procedure is removed, such that implantation time and patient trauma are reduced. The present invention fulfills this and other needs, and addresses other deficiencies of prior art implementations and techniques.

### SUMMARY OF THE INVENTION

The present invention is directed to an apparatus for stabilizing an implantable device having a lumen. In one embodiment of the present invention, the apparatus includes a stylet having an elongated body and an enlarged portion at a distal end of the stylet. The enlarged portion is dimensioned to pass through at least part of the lumen of the implantable device. A sleeve is disposed along the stylet. The stylet and the sleeve are relatively movable so that a distal portion of the sleeve creates an interference between the enlarged portion of the stylet and the lumen of the implantable device. The apparatus also includes a proximal device having a first part connected to the sleeve and a second part connected to the stylet. The proximal device providing controllable relative motion between the sleeve and the stylet.

In another embodiment of the present invention, a method of implanting an implantable device in a destination vessel involves advancing a guide apparatus so that a distal tip of the guide apparatus is near the destination vessel. The implantable device is advanced along the guide apparatus so that a distal end of the implantable device is positioned in the destination vessel. A stylet having an enlarged distal portion is advanced through a lumen of the implantable device so that the enlarged distal portion of the stylet is located at a distal portion of the implantable device. The implantable device is stabilized by engaging a sleeve between the enlarged distal portion of the stylet and the open lumen of the implantable device to fixably couple the stylet and the implantable device, after which the guide apparatus is withdrawn.

### BRIEF DESCRIPTION OF THE DRAWINGS

FIG. 1 is a side view of a stabilizing apparatus according to embodiments of the present invention;

FIG. 2 is an exploded view of a proximal device of the apparatus according to embodiments of the present invention;

FIG. 3A is a cross-sectional view of the proximal device in a locked orientation according to embodiments of the present invention;

FIG. 3B is a cross-sectional view of the proximal device of FIG. 3A in an unlocked orientation;

FIG. 3C is a cross-sectional view of the proximal device of FIG. 3A in an extended unlocked orientation;

FIG. 4 is a side view of a stylet according to embodiments of the present invention;

FIG. 5 is a cross sectional view of an implantable device with the distal end of the stabilizing apparatus in a lumen of the implantable device according to embodiments of the present invention;

FIG. 6A is a side view of the stabilizing apparatus showing an unlocked orientation of the distal end;

FIG. 6B is a side view of the stabilizing apparatus showing an extended unlocked orientation of the distal end;

FIG. 6C is a side view of the stabilizing apparatus showing a locked orientation of the distal end; and

FIG. 7 is a side view of an alternate configuration of a stabilizing apparatus according to embodiments of the present invention.

While the invention is amenable to various modifications and alternative forms, specifics thereof have been shown by way of example in the drawings and will be described in detail herein. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

The present invention is directed to an apparatus used for stabilizing an implantable medical device during implantation procedures. In general, the apparatus is configured to fit inside an implantable device with an inner lumen, such as an over-the-wire cardiac pacing lead. After the lead is implanted in the target vessel of the body, the stabilizing apparatus is used to keep the lead in place while operations are performed that might dislodge the lead. Those operations may include the withdrawal of a guide apparatus used to assist in positioning the lead. Such guide apparatus typically include guide catheters, although other apparatus such as guide wires may also be used to assist in positioning implantable devices.

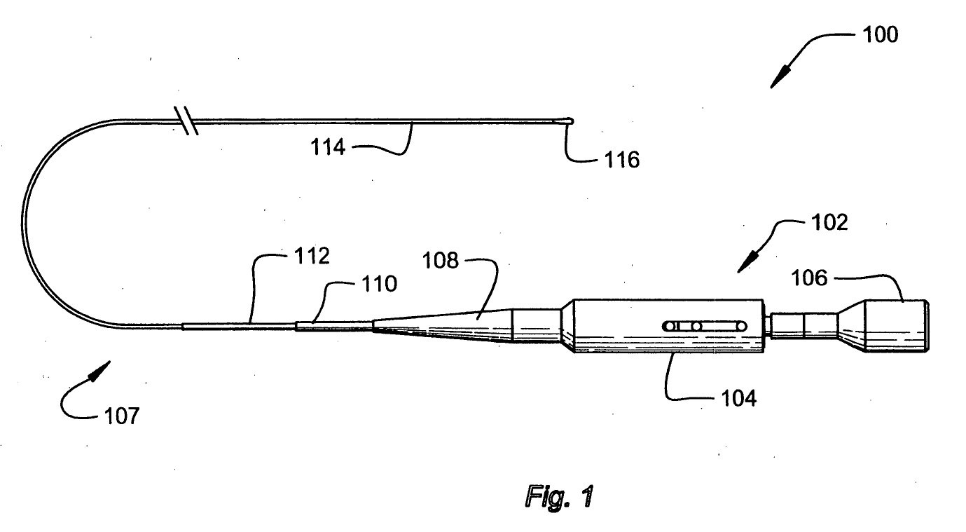

In reference now to FIG. 1, a stabilizing apparatus 100 is illustrated according to embodiments of the present invention. The apparatus 100 includes a proximal device 102 that allows for manipulation of the apparatus 100. The proximal device 102 includes a housing 104 and a slidable handle 106. The housing 104 and handle 106 can be formed from ABS plastic, although other plastic and/or metallic materials may be appropriate.

The housing 104 provides a convenient grip for the apparatus 100, as well providing an attachment for a distal sleeve portion 107 that includes a strain relief 108, a stiffener 110, a hard stop 112, and a sheath 114. The distal sleeve portion 107 contains a stylet 116 (or “finishing wire”) that is slidable within the sheath 114. The stylet 116 includes a bulbous, distal tip 116 that can be drawn into the sheath 114 for locking the apparatus 100 within an implantable device.

The stylet's proximal end is coupled to the handle 106. By sliding the handle 106 in a proximal direction relative to the housing 104, a clinician is able to retract the tip of stylet 116 within the sheath 114, thereby expanding the sheath's diameter at the distal tip of the apparatus 100. This expanded diameter is useful for locking the distal tip of the apparatus 100 in the lumen of an implantable device.

The distal sleeve portion 107 is preferably flexible enough to be introduced through convoluted anatomical passageways, yet provide enough longitudinal stiffness to allow the sleeve portion 107 and stylet 116 to be pushed through an implantable device. The sheath 114 is formed by a relatively stiff and resilient plastic or metal tubing. In particular, a polyamide tube has been found useful in forming the sheath 114. Polyamides are suited to this application due to the polymer's exceptional hardness, good impact strength, resistance to stretching, and abrasion resistance.

The sheath 114 may be fabricated with additional features to provide smoother operation. The sheath 114 may include a thin, lubricous lining (e.g. polytetrafluoroethylene) on the inner surface that helps the stylet 116 to move with less friction within the sheath 114. The sheath 114 may also have an outer coating (e.g. nylon) for smoothness and abrasion resistance.

The sheath 114 is given extra stiffness at the proximal end by the use of the hard stop 112, which in this example is a heat-shrinkable polyolefin tube. Besides stiffening the sheath 114, the hardstop 112 is sized so that it will not fit in the lumen of the implantable device. Therefore, the hardstop 112 can be configured to butt against a proximal part of the implantable device when the stabilizing apparatus 100 is properly positioned within the implantable device. This gives the user tactile feedback that the stabilizing apparatus 100 is properly positioned within the implantable device and prevents the distal tip of the stabilizing apparatus 100 from accidentally exiting the distal end of the implantable device.

The stiffener 110 is placed over the hard stop 112 and also acts to reinforce the sheath 114. The stiffener 110 can be formed from a low-density polyurethane (LDPE) heat-shrink tube. The stiffener 110 improves the dimensional and stiffness transition from the hardstop 112 to the strain relief 108. The strain relief 108 provides bending reinforcement to the distal sleeve portion 107. The strain relief 108 can be molded from a flexible polymer (e.g. Santoprene®) to fit over a portion of the housing 104.

Those skilled in the art will readily appreciate that alternate materials and arrangements may be used to form the distal sleeve portion 107. In general, choice of materials and types of stiffening features will depend on the intended use of the apparatus 100. In general, the distal sleeve portion 107 should be flexible and maneuverable, yet reasonably impervious to rough handling at the proximal end.

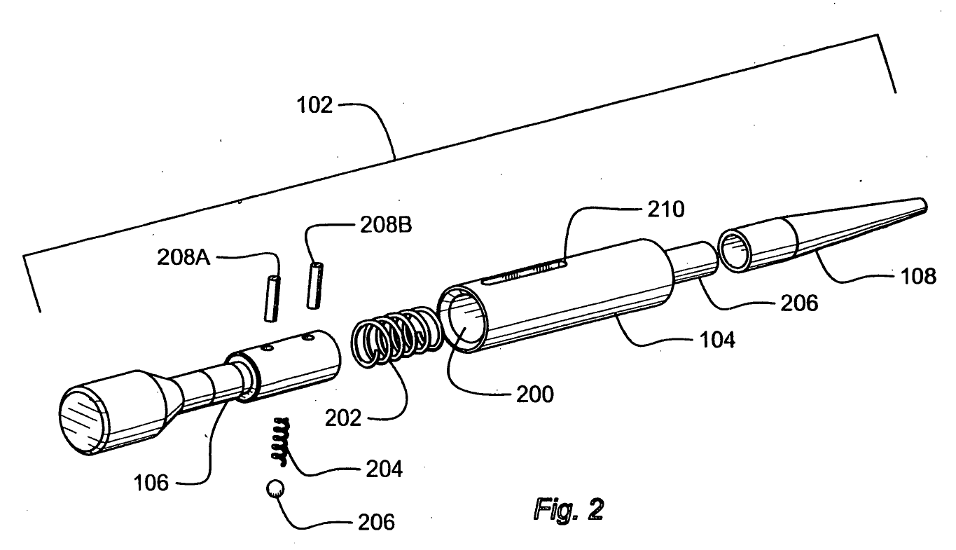

Turning now to FIG. 2, various details of the proximal device 102 are shown. The handle 106 is designed to slidably fit in a bore 200 of the housing 104. The handle 106 is designed to have a locked, unlocked, and extended unlocked orientation within the housing 104. The locked and unlocked orientations of the handle 106 correspond to respective locked and unlocked orientations of the sheath 114 and stylet 116. The extended unlocked orientation allows addition travel of the handle 106 to further assist in disengaging the sheath 114 and stylet 116. A stainless steel spring 202 also fits in the bore 200. The spring 202 provides a return force on the handle 106 from the extended unlocked orientation to the unlocked orientation of the handle 106.

The proximal device 102 includes features to ensure that locking and unlocking of the apparatus occurs reliably, consistently, and with tactile feedback to the user. This is accomplished by having travel limits and locking detents between the handle 106 and the housing 104. The locking detents are provided by a stainless steel ball 204 and an index spring 206 that both fit in a cavity (not shown) of the handle 106. The ball 206 disengageably locks into indentations on the inside surface of the housing bore 200.

The proximal device 102 constrains travel of the handle 106 by including a slot 210 in the housing 104 that interfaces with the pin 208A pressed into the handle 106. The retaining pin 208B is also pressed into the handle 106 to retain the stylet 116 (see FIG. 1). The proximal device 102 may be assembled by placing all attachments for the handle 106 (including the spring 202, locking spring 204, ball 206, retaining pin 208B, and stylet 116) inside the housing bore 200, then installing the pin 208A through the slot 210. In this way, the pin 208A retains the handle 106 in the housing 104.

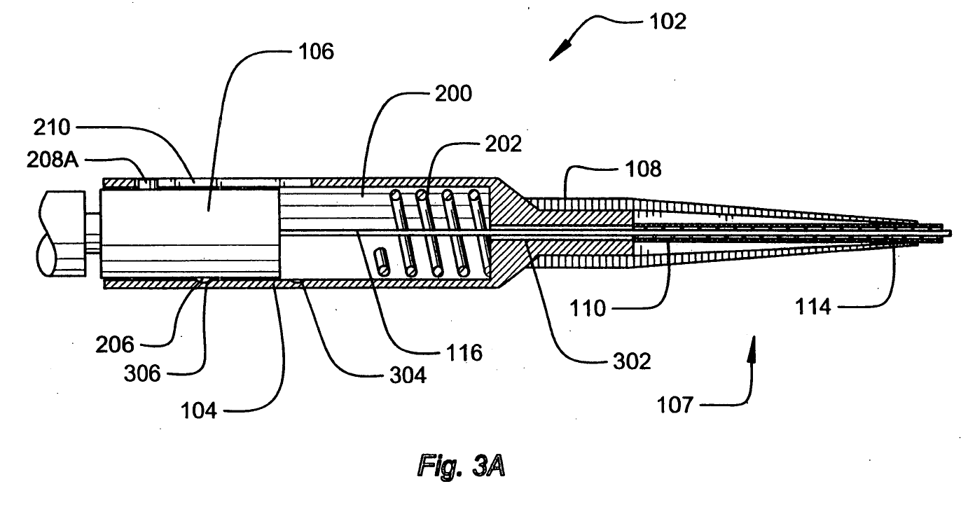

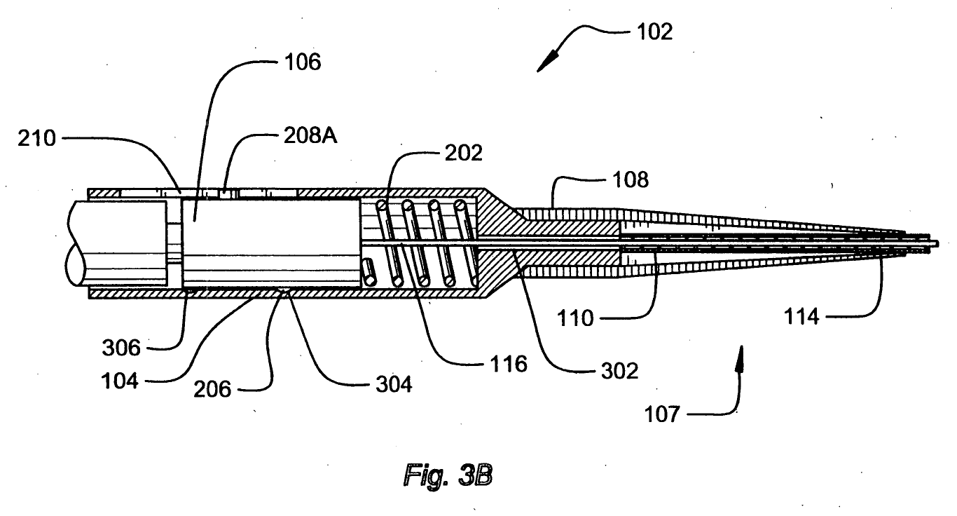

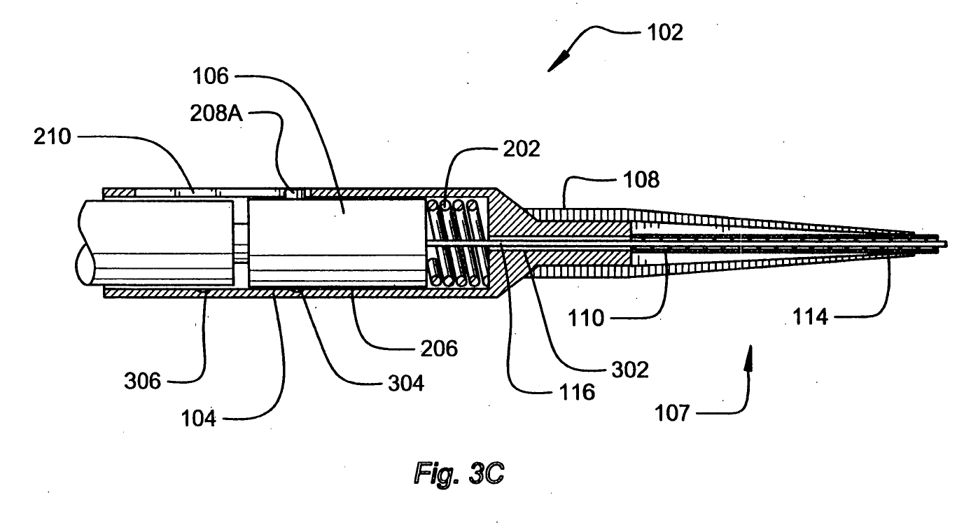

FIG. 3A shows a cross sectional view of the proximal device 102 after assembly. The proximal device 102 is shown in a locked orientation, with the handle 106 retracted to a proximal location. In the locked orientation, the proximal pin 208A is at or near the proximal end of the slot 210 and the ball 206 is seated in a proximal indentation 306.

FIG. 3B shows the unlocked orientation of the proximal device 102, which is obtained by sliding the handle 106 in a distal direction until the ball 206 is seated in the distal indentation 304. In the unlocked orientation, the pin 208A is approximately two thirds of the way to the distal end of the slot 210. Note that the handle 106 may be touching the spring 202 in the unlocked orientation, although the spring 202 is typically not compressed.

FIG. 3C shows the extended unlocked orientation of the proximal device 102. In the extended unlocked position, pin 208A is located at or near the distal end of the slot 210, and the ball 206 is not seated in any indentation. Note that the spring 202 is compressed in the extended unlocked orientation.

FIGS. 3A-C also show the proximal end of the stylet 116 attached to the handle 106. The stylet 116 is fixably coupled to the center of the handle 106 by the retaining pin 208B (not shown). Sliding of the handle 106, therefore, moves the stylet 116 axially within the sheath 114 to provide locking and unlocking of the stylet's distal end.

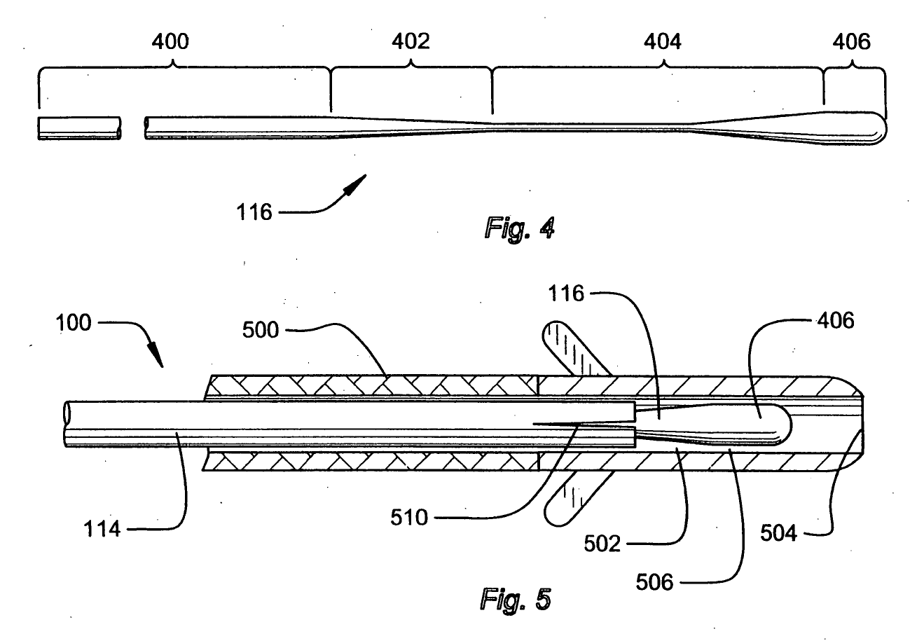

The stylet 116 is shown in greater detail in FIG. 4. The stylet's proximal portion 400 has a substantially constant cross sectional diameter. Near the distal end of the stylet, a taper 402 (or “neck down”) area is formed. The taper 402 transitions to a narrowed portion 404, and the narrowed portion 404 eventually increases in diameter as it joins with a ball-nose distal end 406. The stylet 116 is generally formed from a suitable stainless steel material (e.g. 304V) and should include smooth transitions and contours on all distal features 402, 404, 406.

Although the illustrated embodiment of the stylet 116 shows a ball-nose distal end 406, it will be appreciated that alternate tip shapes may achieve similar results, such as a flattened and broadened tip portion or a tip with an increasing taper leading to a substantially flat distal end. However, the distal end 406 is preferable smooth and atraumatic so that it doesn't damage the sleeve 114 during assembly.

In general, the distal end 406 of the stylet 116 is designed to have larger outer dimensions than the proximal portion 400. This allows the stylet 116 to move easily within the sheath 114, yet will expand the distal end of the sheath 114 when the ball-nose distal end 406 is moved within the sheath 114. The distal arrangement of the stylet 116 and sheath 114 is shown in FIG. 5.

In FIG. 5, an implantable device 500 is shown in cross section with a stylet 116 and sheath 114 deployed within. The implantable device 500 includes a lumen 502. The illustrated lumen 502 is open at the device's distal end 504, thereby allowing the implantable device 500 to be introduced over a guide wire. However, the stabilizing apparatus 100 can also be used in devices without an opening at the distal end 504, as long as the device 500 contains a lumen 502 accessible by the stylet 116.

In FIG. 5, the stabilizing apparatus 100 is shown in an unlocked orientation. In this orientation, the end of the sheath 114 is just behind the enlarged, distal end 406 of the stylet 116. In the unlocked orientation, there is a clearance 506 around the stylet's distal end 406.

The sheath 114 is configured to slide distally over the enlarged end 406 of the stylet 116 and fill the clearance 506. To ease sliding the sheath 114 over the stylet's distal end 406, the sheath 114 may include a distal slot 510 as shown in FIG. 5. The slot 510 allows the sheath 114 to spread at the distal end when moving over the larger diameter part of the stylet 116. The sheath 114 thus fills the clearance 506 and creates an interference between the stylet 116 and the lumen 502 of the implantable device 500. This interference allows the stylet 116 to be used to stabilize the implantable device 500.

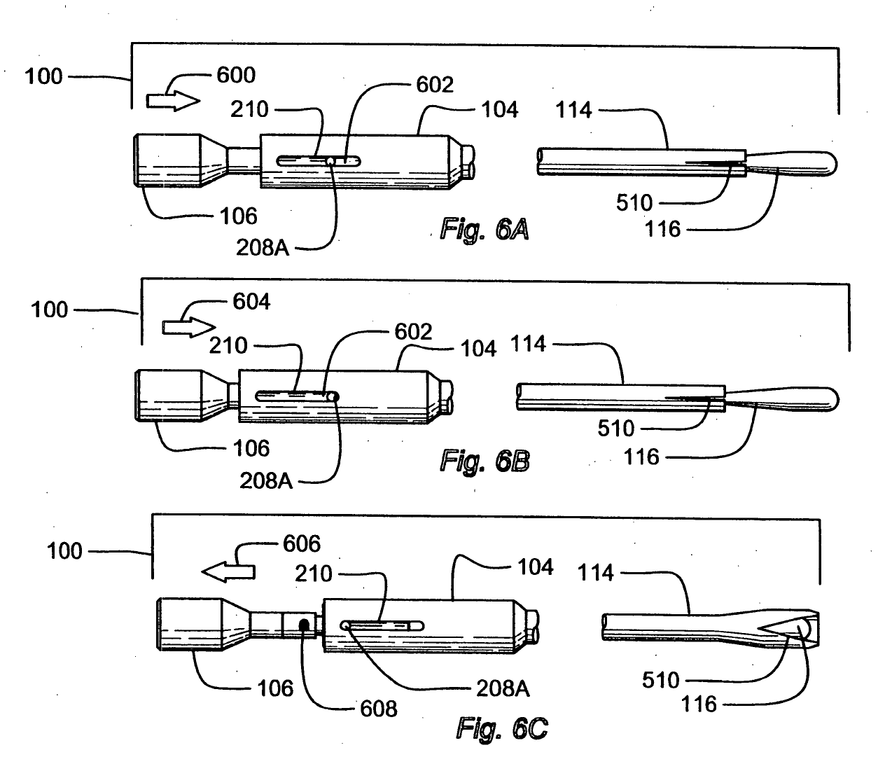

The locking and unlocking of the apparatus 100 is illustrated in FIGS. 6A, 6B, and 6C. In FIG. 6A, the apparatus is in an unlocked orientation. The handle 106 is distally located as indicated by the arrow 600. This orientation of the handle 106 locates the distal end of the stylet 116 past the distal end of the sheath 114. Also note the location of the pin 208A relative to the slot 210 of the housing 104. As seen in the unlocked orientation of FIG. 6A, a gap 602 exists between the pin 208A and the distal end of the slot 210. By leaving this gap 602, the user can push the handle 106 past the detents of the unlocked orientation to the extended unlocked orientation. The extended unlocked orientation is illustrated in FIG. 6B.

In FIG. 6B, the handle 106 has been pushed further in a distal direction as indicated by the arrow 604. This extra distal travel beyond the unlocked orientation of FIG. 6A allows the user to apply additional unlocking force to enhance the unlocking ability of the device.

In FIG. 6C, the handle 106 has been moved in a proximal direction relative to the housing 104, as indicated by the arrow 606. This is the locked orientation of the apparatus 100. The handle 106 pulls the distal end of the stylet 116 into the sheath 114. This enlarges the distal end of the sheath 114, which can then engage the lumen of an implantable device. The handle 106 may also include an indicator 608 that is visible when the apparatus 100 is in the locked position. The indicator 608, along with detent features described in relation to FIGS. 2 and 3, advantageously give the user positive verification of the locked or unlocked state of the apparatus 100.

In general, the stabilizing apparatus 100 is sized according to the dimensions of the implantable device with which it is intended to be used. In one example, a stabilizing apparatus 100 is usable with a cardiac lead having a nominal lumen diameter of 0.022 inches. The stylet 116 in this apparatus 100 is formed from a 60-inch-long, 304V stainless steel wire having a proximal diameter of 0.0113 inches and a 0.0197 inch diameter ball-nose end. The sheath 114 for use with this stylet 116 includes an outer diameter of 0.0195 inches and an inner diameter of 0.0130 inches. The slot 510 length is 0.177 inches long.

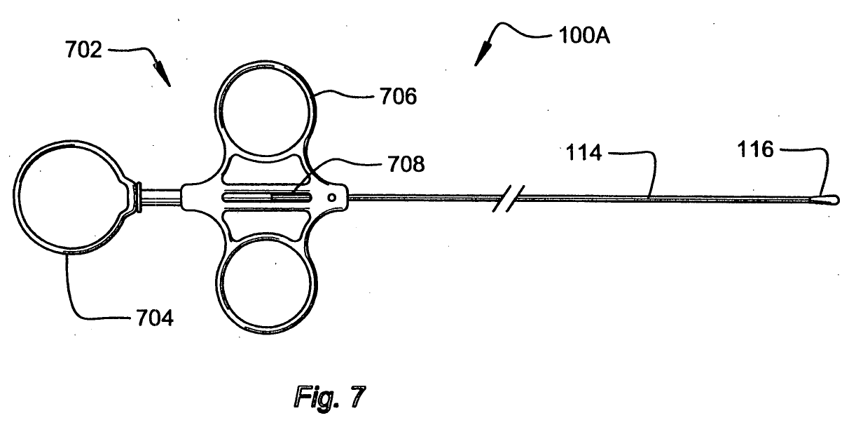

FIG. 7 shows an alternate arrangement of a stabilizing apparatus 100A according to an alternate embodiment of the present invention. In this apparatus, a proximal device 702 includes a ring handle 704 slidably deployed in a dual-ring housing 706 The rings on the handle 704 and housing 706 allow the proximal device 702 to be locked and unlocked with one hand. As with the previous embodiments, the sheath 114 is coupled to the housing 706, and the stylet 116 is coupled to the handle 704. The housing 706 includes a slot 708 that can be used for interfacing with handle stop features (e.g. pins) and/or for providing a visual indication of locked and unlocked orientations.

It will, of course, be understood that various modifications and additions can be made to the embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A method of placing an implantable device in a cardiac vessel, comprising:
positioning the implantable device so that a distal end of the implantable device is positioned in the cardiac vessel;
positioning a stylet having an enlarged distal portion within a lumen of the implantable device so that the enlarged distal portion of the stylet is located at a distal portion of the implantable device;
positioning a sleeve within the implantable device and in proximity to the enlarged distal portion of the stylet; and
stabilizing the implantable device by selective engagement and disengagement between the enlarged distal portion of the stylet and the sleeve.

2. The method of claim 1, wherein engagement between the enlarged distal portion of the stylet and the sleeve comprises:
advancing the sleeve over the stylet and to a position proximal of the enlarged distal portion of the stylet; and
retracting the stylet to draw the enlarged distal portion of the stylet into the sleeve such that the sleeve is urged into coupling engagement with a surface of the lumen of the implantable device.

3. The method of claim 1, wherein disengagement between the enlarged distal portion of the stylet and the sleeve comprises advancing the stylet in a distal direction relative to the sleeve to free the enlarged distal portion of the stylet from the sleeve.

4. The method of claim 1, wherein the sleeve comprises a slot at a distal end of the sleeve configured to facilitate drawing of the enlarged distal portion of the stylet into the sleeve.

5. The method of claim 1, wherein the sleeve comprises a slot at a distal end of the sleeve, the slot allowing the distal end of the sleeve to spread and engage a surface of the lumen of the implantable device when the enlarged distal portion of the stylet is received by the sleeve.

6. The method of claim 1, further comprising selectively locking and unlocking proximal end portions of the stylet and the sleeve.

7. The method of claim 1, further comprising selectively locking and unlocking orientations of the stylet and the sleeve.

8. The method of claim 7, wherein the locking orientations of the stylet and the sleeve correspond to engaged relationships between the stylet and a surface of the lumen of the implantable device, and the unlocking orientations correspond to disengaged relationships between the stylet and the lumen surface of the implantable device.

9. The method of claim 1, wherein the cardiac vessel is a left heart vessel.

10. The method of claim 1, wherein the lumen of the implantable device is an open lumen.

11. The method of claim 1, wherein the lumen of the implantable device is a closed lumen.

12. The method of claim 1, wherein the sleeve comprises a polyamide tube.

13. A method of placing an implantable device in a cardiac vessel, comprising:
positioning the implantable device so that a distal end of the implantable device is positioned in the cardiac vessel;
advancing a stylet having an enlarged distal portion through a lumen of the implantable device so that the enlarged distal portion of the stylet is located at a distal portion of the implantable device;
advancing the sleeve over the stylet and to a position proximal of the enlarged distal portion of the stylet;
retracting the stylet to draw the enlarged distal portion of the stylet into the sleeve such that the sleeve is urged into coupling engagement with the lumen surface of the implantable device to stabilize the implantable device; and
advancing the stylet relative to the sleeve to free the enlarged distal portion of the stylet from the sleeve to allow relative movement between the stylet and the implantable device.

14. The method of claim 13, wherein the sleeve comprises a slot at a distal end of the sleeve configured to facilitate drawing of the enlarged distal portion of the stylet into the sleeve.

15. The method of claim 13, wherein the sleeve comprises a slot at a distal end of the sleeve, the slot allowing the distal end of the sleeve to spread and engage the lumen surface of the implantable device when the enlarged distal portion of the stylet is received by the sleeve.

16. The method of claim 13, further comprising selectively locking and unlocking proximal end portions of the stylet and the sleeve.

17. The method of claim 13, further comprising selectively locking and unlocking orientations of the stylet and the sleeve.

18. The method of claim 13, wherein the cardiac vessel is a left heart vessel.

19. The method of claim 13, wherein the lumen of the implantable device is an open lumen.

20. The method of claim 13, wherein the lumen of the implantable device is a closed lumen.
